(19) United States
(12) Patent Application Publication
(10) Pub. No.: US 2009/0099452 A1
(43) Pub. Date: Apr. 16, 2009

(54) ULTRASONIC IMAGING APPARATUS

(76) Inventor: Hiroshi Hashimoto, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/251,616

(22) Filed: Oct. 15, 2008

(30) Foreign Application Priority Data
Oct. 15, 2007 (JP) 2007-267446

(51) Int. Cl. A61B 8/14

(52) U.S. Cl. 600/443

(57) ABSTRACT

An ultrasonic imaging apparatus includes an image acquisition device for administering a contrast agent and acquiring B-mode image information, an image storing device for storing the B-mode image information during a predetermined imaging time range from after the administration, a first maximum intensity projection device for forming first maximum intensity projection image information using the B-mode image information acquired in a first time range contained in the imaging time range, a second maximum intensity projection device for forming second maximum intensity projection image information using the B-mode image information acquired in a second time range contained in the first time range, a second image formation device for forming a second image based on the second maximum intensity projection image information, an overlaid image generation device for generating an overlaid image in which the second image is overlaid on a first image of the first maximum intensity projection image information.

### CROSS REFERENCE TO RELATED APPLICATIONS

This application claims the benefit of Japanese Patent Application No. 2007-267446 filed Oct. 15, 2007, which is hereby incorporated by reference in its entirety.

### BACKGROUND OF THE INVENTION

The subject matter disclosed herein relates to an ultrasonic imaging apparatus which images a contrast agent administered to a subject and observes a time change in the contrast agent that permeates through an imaging area thereof.

It has recently been practised to administer a contrast agent to a subject and observe B-mode image in an imaging area, about the blood vessels or a tissue portion or the like through which the contrast agent has permeated, using an ultrasonic imaging apparatus. The B-mode image indicates the manner in which a distribution of the contrast agent in the imaging area changes with time. Upon this imaging, maximum intensity projection (abbreviated as “MIP”) using acquired B-mode images is conducted to make it easier for an operator to observe the distribution of the contrast agent, which changes momentarily.

The maximum intensity projection compares pixel values lying in the same pixel position between a plurality of acquired B-mode images, determines the maximum pixel value of these pixel values and forms maximum intensity projection image information comprised of the maximum pixel value. In the B-mode image information, the contrast agent is extracted as a high brightness or intensity area, and the process of the position and intensity of this high-brightness area being changed is observed. Thus, the maximum intensity projection image results in one in which a contrast agent distribution at the acquisition of the B-mode images is projected onto one image together with the past distribution status.

According to the above background art, however, the maximum intensity projection image could not provide visual comparisons between a time change in the distribution of the contrast agent and all permeation processes including the process that the contrast agent will permeate after the acquisition of the maximum intensity projection image. That is, the maximum intensity projection image assumes the image in which the current and past distributions of contrast agent are overlaid, where it is formed in time with the acquisition thereof in real time. This cannot be compared with other portion or region, particularly, a region through which the contrast agent will permeate.

Now, while the time change indicated by the contrast agent distribution in the subject brings about important information clinically, the contrast-agent permeating process is not understood by itself but understood more clearly by visually comparing other region, particularly, a region through which the contrast agent permeated in the past, and a region or the like through which the contrast agent will permeate and making relative comparisons of shapes or the like with these regions.

In the case of the liver, for example, the contrast agent flows therein from the artery thereof and migrates into the portal vein thereof. At this time, the grasping of the behavior of the contrast agent in the artery and the behavior of the contrast agent in the portal vein corresponding to the destination along with the difference in time phase between them yields various information in diagnosing lesions.

In view of these, it is of importance of how an ultrasonic imaging apparatus having image information that enables a visual comparison of the time change in the maximum intensity projection image inclusive of the region through which the contrast agent permeates after the acquisition of the maximum intensity projection image is realized.

### BRIEF DESCRIPTION OF THE INVENTION

It is desirable that the problem described previously is solved.

An ultrasonic imaging apparatus according to the invention of a first aspect includes an image acquisition device for administering a contrast agent to a subject and acquiring B-mode image information in an imaging area of the subject through which the contrast agent permeates, an image storing device for storing a plurality of the B-mode image information changed with the permeation therein during a predetermined imaging time range from after the administration, a first maximum intensity projection device for forming first maximum intensity projection image information by maximum intensity projection using the B-mode image information acquired in a first time range contained in the imaging time range, a second maximum intensity projection device for forming second maximum intensity projection image information by maximum intensity projection using the B-mode image information acquired in a second time range contained in the first time range, a second image formation device for forming a second image based on the second maximum intensity projection image information, an overlaid image generation device for generating an overlaid image in which the second image is overlaid on a first image of the first maximum intensity projection image information, and a display device for displaying the overlaid image.

In the invention according to the first aspect, the first maximum intensity projection device forms first maximum intensity projection image information by maximum intensity projection using B-mode image information acquired in a first time range contained in an imaging time range. The second maximum intensity projection device forms second maximum intensity projection image information by maximum intensity projection using B-mode image information acquired in a second time range contained in the first time range and generates and displays an overlaid image in which a second image based on the second maximum intensity projection image information are overlaid on a first image of the first maximum intensity projection image information.

An ultrasonic imaging apparatus according to the invention of a second aspect is provided wherein in the ultrasonic imaging apparatus described in the first aspect, an input unit is provided which inputs designation formation on the imaging time range, the first time range and the second time range.

In the invention of the second aspect, the input unit performs time designation for an imaging time range, a first time range and a second time range.

An ultrasonic imaging apparatus according to the invention of a third aspect is provided wherein in the ultrasonic imaging apparatus described in the second aspect, the input unit has a time range designation key which inputs the designation information on the imaging time range, the first time range and the second time range.

An ultrasonic imaging apparatus according to the invention of a fourth aspect is provided wherein in the ultrasonic imaging apparatus described in the third aspect, the time range designation key inputs frame number information or acquisition time information of the B-mode image information constituting the designation information while displaying the B-mode image information for the imaging time range on the display device.

In the invention of the fourth aspect, first and second time ranges are easily designated by frame numbers or acquisition times while referring to B-mode image information in an imaging time range.

An ultrasonic imaging apparatus according to the invention of a fifth aspect is provided wherein in the ultrasonic imaging apparatus described in any one of the second through fourth aspects, the designation information includes start time information and end time information for the imaging time range.

An ultrasonic imaging apparatus according to the invention of a sixth aspect is provided wherein in the ultrasonic imaging apparatus described in any one of the second through fifth aspects, the designation information includes start time information and end time information for the first time range.

An ultrasonic imaging apparatus according to the invention of a seventh aspect is provided wherein in the ultrasonic imaging apparatus described in any one of the second through sixth aspects, the designation information includes start time information and end time information for the second time range.

An ultrasonic imaging apparatus according to the invention of an eighth aspect is provided wherein in the ultrasonic imaging apparatus described in any one of the first through seventh aspects, the second image is an image of the second maximum intensity projection image information.

In the invention of the eighth aspect, an image of second maximum intensity projection image information is overlaid on a first image as it is.

An ultrasonic imaging apparatus according to the invention of a ninth aspect is provided wherein in the ultrasonic imaging apparatus described in any one of the first through seventh aspects, the second image formation device includes a contrast table in which pixel values and hues of image information are associated with one another.

An ultrasonic imaging apparatus according to the invention of a tenth aspect is provided wherein in the ultrasonic imaging apparatus described in the ninth aspect, the second image is a colour image in which a pixel value of the second maximum intensity projection image information is associated with a hue, based on the contrast table.

In the invention of the tenth aspect, a second image is displayed in colour.

An ultrasonic imaging apparatus according to the invention of an eleventh aspect is provided wherein in the ultrasonic imaging apparatus described in any one of the first through tenth aspects, the input unit includes an overlay ratio designation key which inputs overlay ratio information indicative of a ratio in brightness between the first image and the second image at the execution of the overlay.

An ultrasonic imaging apparatus according to the invention of a twelfth aspect is provided wherein in the ultrasonic imaging apparatus described in the eleventh aspect, the overlaid image formation device changes pixel values of the first and second images to overlay the first and second images, based on the overlay ratio information.

In the invention of the twelfth aspect, overlaid image formation device changes an overlay ratio between first and second images to easy to see them.

An ultrasonic imaging apparatus according to the invention of a thirteenth aspect is provided wherein in the ultrasonic imaging apparatus described in any one of the first through twelfth aspects, the second time range includes a plurality of time ranges set with respect to the one first time range.

In the invention of the thirteenth aspect, second images different in time range are acquired.

An ultrasonic imaging apparatus according to the invention of a fourteenth aspect is provided wherein in the ultrasonic imaging apparatus described in the thirteenth aspect, the second image formation device has a plurality of the contrast tables including different hues and associates the contrast tables having the different hues with one another every second maximum intensity projection image information for the set time ranges thereby to form second images different in hue.

An ultrasonic imaging apparatus according to the invention of a fifteenth aspect is provided wherein in the ultrasonic imaging apparatus described in any one of the first through fourteenth aspects, the second maximum intensity projection device changes the second time range with time stepwise from zero to form second maximum intensity projection image information every step.

An ultrasonic imaging apparatus according to the invention of a sixteenth aspect is provided wherein in the ultrasonic imaging apparatus described in the fifteenth aspect, the second image formation device forms a second image, based on the second maximum intensity projection image information for each step referred to above, and the overlaid image formation device generates an overlaid image using the second image for each step referred to above.

An ultrasonic imaging apparatus according to the invention of a seventeenth aspect is provided wherein in the ultrasonic imaging apparatus described in the sixteenth aspect, the display device cine-displays the overlaid image for each step referred to above in sync with the change with the time.

In the invention of the seventeenth aspect, a second image contained in an overlaid image is changed with time.

An ultrasonic imaging apparatus according to the invention of an eighteenth aspect includes an image acquisition device for administering a contrast agent to a subject and acquiring B-mode image information in an imaging area of the subject through which the contrast agent permeates, an image storing device for storing a plurality of the B-mode image information changed with the permeation therein during a predetermined imaging time range from after the administration, a first maximum intensity projection device for forming first maximum intensity projection image information by maximum intensity projection using the B-mode image information acquired in a first time range contained in the imaging time range, a second maximum intensity projection device for changing a second time range contained in the first time range with time stepwise from zero and forming second maximum intensity projection image information by maximum intensity projection using the B-mode image information acquired in the second time range, for each step, a second image formation device for forming a second image based on the second maximum intensity projection image information at each step referred to above, an overlaid image generation device for generating an overlaid image in which the second image is overlaid on a first image of the first maximum intensity projection image information, at each step referred to above, and a display device for displaying the overlaid image at each step referred to above.

According to the invention, an operator is able to easily make a contradistinction between a second image and a first image overlaid thereon through an overlaid image of the first and second images subjected to maximum intensity projection, make it easy to understand a time change in contrast agent and easily make a contradistinction to all permeation processes of the contrast agent, which occur during the time subsequent to the time required to acquire the second image.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an overall construction of an ultrasonic imaging apparatus.

FIG. 2 is an explanatory diagram illustrating an operation panel included in an input unit of the ultrasonic imaging apparatus.

FIG. 3 is a block diagram showing a detailed construction of a controller and an image display controller.

FIG. 4 is an explanatory diagram depicting one example of a contrast table that causes pixel values and hues to correspond.

FIG. 5 is a flowchart showing the operation of the controller.

FIG. 6 is an explanatory diagram depicting one example illustrative of B-mode images acquired within a first time image.

FIG. 7 is an explanatory diagram showing one example of an MIP completed image.

FIG. 8 is an explanatory diagram illustrating one example of an MIP progress image.

FIG. 9 is an explanatory diagram depicting one example of an overlaid image.

FIG. 10 is an explanatory diagram showing one example of a cine display in which overlaid images are changed in time order.

### DETAILED DESCRIPTION OF THE INVENTION

Various embodiments of an ultrasonic imaging apparatus according to the invention will be explained below with reference to the accompanying drawings. Incidentally, the invention is not limited thereby.

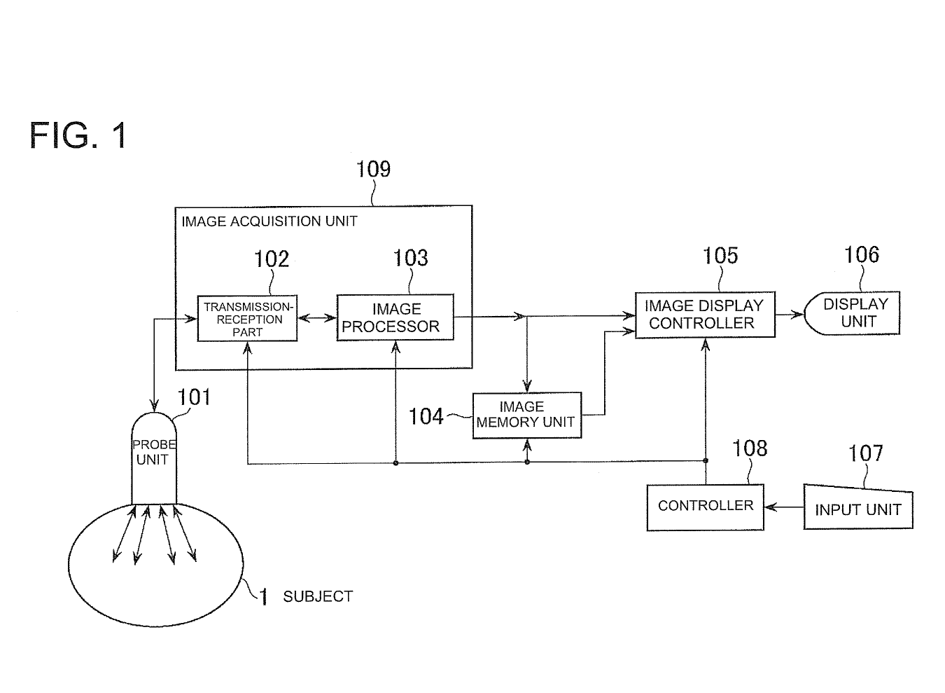

An overall construction of an ultrasonic imaging apparatus according to a first embodiment will first be explained. FIG. 1 is a block diagram showing the overall construction of the ultrasonic imaging apparatus according to the embodiment 1. The ultrasonic imaging apparatus includes a probe unit 101, an image acquisition unit 109, an image memory unit 104 that constitutes image storing device, an image display controller 105, a display unit 106 that constitutes display device, an input unit 107 and a controller 108.

The probe unit 101 repeatedly transmits ultrasound in a specific direction of an imaged section of a region, i.e., a subject 1 for transmitting and receiving the ultrasound and receives ultrasonic signals reflected from inside the subject 1 as time-series sound rays. Concurrently with it, the probe unit 101 performs electronic scanning while the directions to transmit the ultrasound are being switched sequentially. Incidentally, although not shown in the figure, piezoelectric elements are arranged in the probe unit 101 in array form.

The transmission-reception part 102 is connected to the probe unit 101 by a coaxial cable and performs the generation of an electric signal for driving each piezoelectric element of the probe unit 101 and first-stage amplification of each ultrasonic signal received thereat.

The image processor 103 performs the formation of an electric signal for driving the transmission-reception part 102 and the formation of B-mode image information or the like from the ultrasonic signal amplified by the transmission-reception part 102. Particularly when a contrast agent is administered to the subject 1, the image processor 103 performs contrast mode processing for generating a contrast mode image such as a B-mode image in real time.

The image processor 103 delays a transmission signal and brings a focus depth position into focus in the case of, for example, ultrasonic transmission as specific contents of processing. In the case of ultrasonic reception, the image processor 103 performs delay/addition processing of a received ultrasonic signal, A/D (analog/digital) conversion processing, processing for writing post-conversion digital information to the image memory unit 104 as B-mode image information, and the like.

The image memory unit 104 is an image memory for storing each B-mode image information or the like generated by the contrast mode processing. In particular, the image memory unit 104 stores the B-mode image information that changes in time therein with a frame constituting one frame of B-mode image formation in an imaging area. This storage is done over an imaging time range between the instant when the contrast agent has been administered to the subject I and the instant when the contrast agent circulates within the subject 1 and reaches the imaging area and further the contrast agent passes through the imaging area or is absorbed by a tissue portion. Incidentally, the imaging time range is set by the input of designated information using keys or the like of the input unit 107, which is conducted by an operator while the B-mode image is being observed. Upon this storage, the acquired B-mode image information is stored in the image memory unit 104 together with time information for its acquisition.

The image display controller 105 performs display frame rate conversion of the B-mode image information or the like generated at the image processor 103, colour display control and shape/position control of a displayed image. The image display controller 105 also performs the display of ROI (region of interest) indicative of a region of interest on the displayed image such as the B-mode image information, etc.

The display unit 106 displays image information outputted from the image display controller 105 visually to the operator by using a CRT (cathode ray tube) or an LCD (liquid crystal display) or the like. The display unit 106 is also capable of performing a color display in accordance with instructions issued from the image display controller 105.

The controller 108 controls the operations of the above respective parts of the ultrasonic imaging apparatus, based on an operation input signal supplied from the input unit 107 and the program and data stored in advance to cause the display unit 106 to display the B-mode image or the like.

The input unit 107 consists of a keyboard and a pointing device or the like. A key for an operation or control input signal for selecting an imaging mode such as B-mode imaging by the operator, a key for designating first and second time ranges for performing maximum intensity projection, a key for inputting an overlay ratio, etc. exist therein.

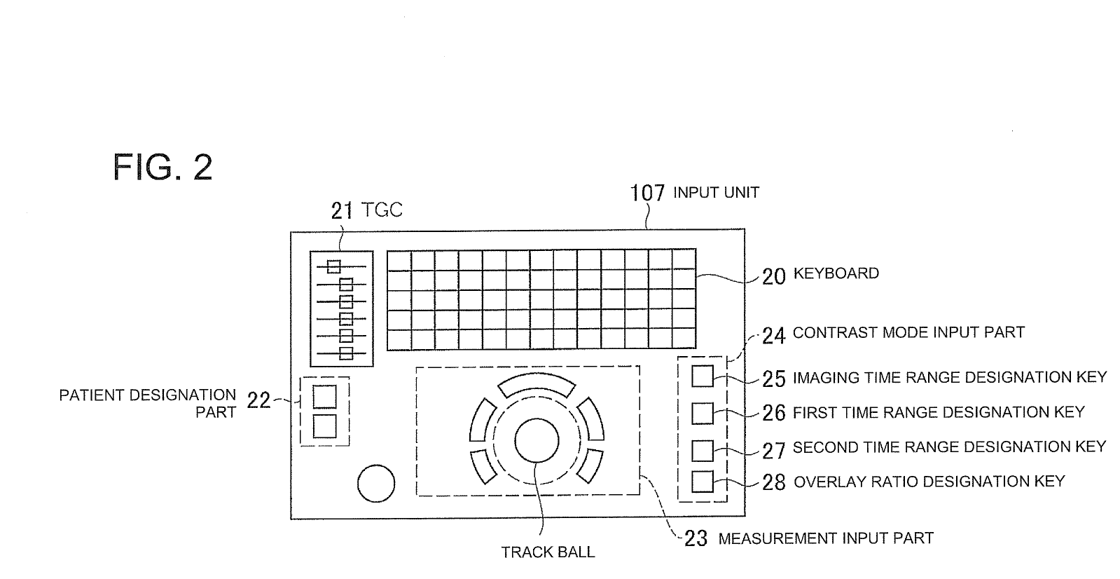

FIG. 2 is a diagram showing one example of a panel of the input unit 107. The input unit 107 includes a keyboard 20, a TGC (Time Gain Controller) 21, a patient designation part 22 including a New Patient Key or the like, a measurement input part 23 including a track ball corresponding to a pointing device, the setting of ROI, etc., and a contrast mode input part 24 including setting keys at the use of the contrast agent.

The contrast mode input part 24 includes an imaging time range designation key 25, a first time range designation key 26 and a second time range designation key 27, an overlay ratio designation key 28, etc. The imaging time range designation key 25 is pressed by the operator after the contrast agent has been administered to the subject 1 to start the storage of the acquired B-mode image information into the image memory unit 104. When the contrast agent exists in the blood vessels and has passed through the imaging area of the subject 1 or when the contrast agent is absorbed by a tissue portion and a contrast agent distribution is brought to a stationary state, the imaging time range designation key 25 is pressed again by the operator to stop the storage of the acquired B-mode image information into the image memory unit 104.

The first time range designation key 26 and the second time range designation key 27 are keys for inputting information for designating a first time range and a second time range. Using the keyboard 20 or the like after these keys have been pressed, start time information and end time information corresponding to the information for designating the first time range and the second time range are inputted sequentially.

The overlay ratio designation key 28 is a key for, when an elapsed or progress image corresponding to a second image and a completed image corresponding to a first image both are displayed to overlap each other, inputting overlay ratio information indicative of a ratio in brightness between the displayed images. For example, the input of a numerical value indicative of an overlay ratio by the keyboard 20 is performed after the overlay ratio designation key 28 has been pressed.

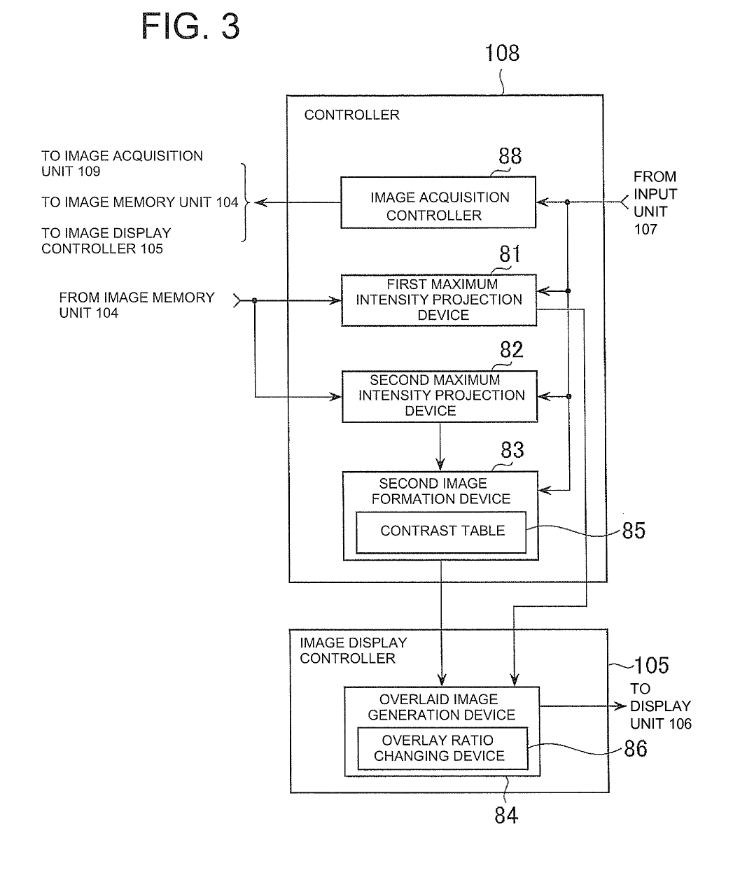

FIG. 3 is a block diagram showing a detailed configuration of the controller 108 and the image display controller 105. The controller 108 includes an image acquisition control unit 88, a first maximum intensity projection device 81, a second maximum intensity projection device 82 and a second image formation device 83. The image display controller 105 includes an overlaid image generation device 84.

The image acquisition control unit 88 performs an ultrasonic scan, based on scan information such as the imaging mode designation information sent from the input unit 107 to acquire or obtain B-mode image information. In particular, the image acquisition control unit 88 recognizes through the designation information of the imaging time range designation key 25 from the input unit 107 that the contrast agent has been administered to the subject 1 and starts the storage of the B-mode image information into the image memory unit 104.

The first maximum intensity projection device 81 reads the B-mode image information on the plural frames stored in the image memory unit 104 in accordance with the designation information about the first time range designated from the input unit 107 and compares pixel values at the same pixel position between these frames of B-mode image information to determine the maximum pixel value. The first maximum intensity projection device 81 forms maximum intensity projection completed image information (hereinafter called MIP completed image information) corresponding to new single frame of first maximum intensity projection image information with the maximum pixel value as a pixel value.

The second maximum intensity projection device 82 compares pixel values at the same pixel position between the frames using the B-mode image information of the plural frames stored in the image memory unit 104, which are designated by the second time range from the input unit 107 thereby to obtain the maximum pixel value. The second maximum intensity projection device 82 forms maximum intensity projection elapsed or progress image information (hereinafter called MIP elapsed or progress image information) corresponding to new single frame of second maximum intensity projection image information with the maximum pixel value as a pixel value.

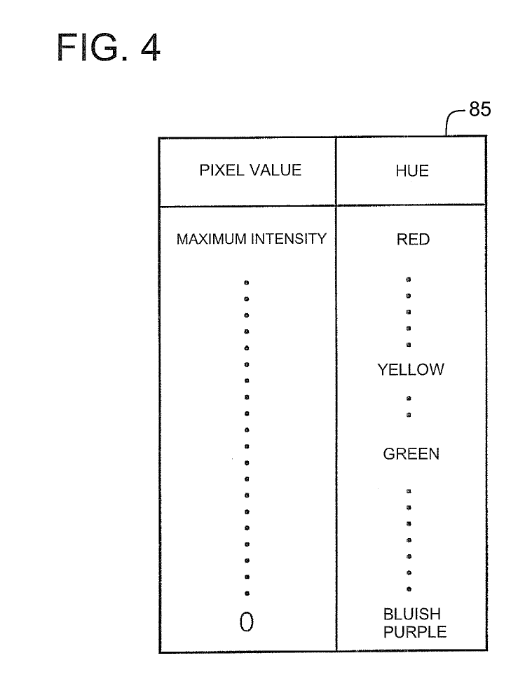

The second image formation device 83 forms an elapsed or progress image corresponding to a second image using the MIP progress image information formed by the second maximum intensity projection device 82. When the progress image is not designated in particular from the input unit 107, the pixel value of the MIP progress image information is used as it is. The second image formation device 83 includes a contrast table in which pixel values and hues are associated with one another. When the designation of a colour display is made from the input unit 107, the pixel value of the MIP progress image information is set as the value of a code associated with the hue. FIG. 4 is an explanatory diagram showing one example of the contrast table 85. The contrast table 85 is one example of a table in which 0 to the maximum value or intensity corresponding to the pixel values are associated with the hues from the blue purple to the red lying in a visible-light range. Incidentally, the hue is represented by characters of the blue purple to red to provide easy understanding in FIG. 4. However, the corresponding values of codes are actually associated with the hues of the blue purple to red. When the code is transmitted to the image display controller 105, the corresponding hue is represented at a pixel position of the display unit 106.

Referring back to FIG. 3, the overlaid image generation device 84 includes an overlay ratio changing device 86 and displays the completed image of the MIP completed image information and the progress image of the MIP progress image information in overlapped form at the same position of the display unit 106 as an overlaid image. The overlay ratio changing device 86 changes the brightness values of the completed image and the progress image, based on an overlay ratio inputted from the input unit 107. When, the overlay ratio is defined as the brightness value of the progress image where the brightness value of the completed image is set as 1, the pixel value of the progress image is set to a value multiplied by the overlay ratio. Since the completed image and the progress image are displayed on the display unit 106 in overlapped form at the same position, the overlaid image is one obtained by adding the completed image corresponding to the first image and the progress image corresponding to the second image.

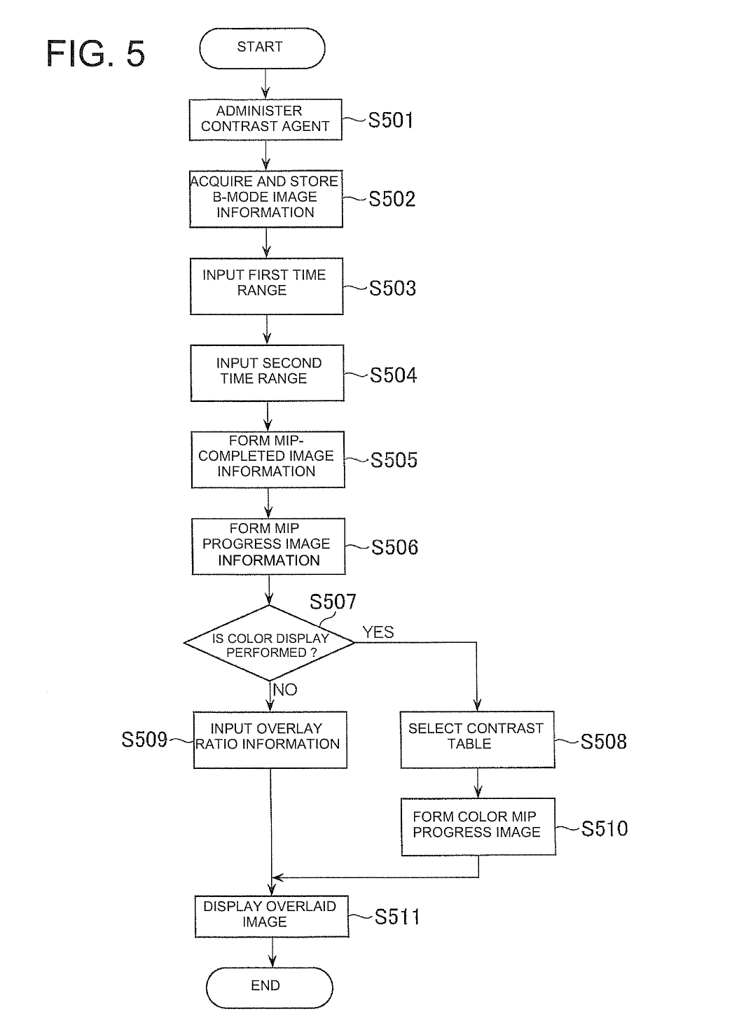

The operation of the controller 108 will next be explained using FIG. 5. FIG. 5 is a flowchart showing the operation of the controller 108. An operator first administers a contrast agent to the subject 1 (Step S501). Then, the operator brings the probe unit 101 into intimate contact with the subject 1 and acquires B-mode image information in a target imaging area. At the same time, the operator designates an imaging time range through the imaging time range designation key 25 and performs the storage of the acquired B-mode image information into the image memory unit 104 (Step S502). Here, the imaging time range designated by the operator is ended after, for example, the entry of the contrast agent is started from approximately immediately after the contrast agent has been administered and the contrast agent in the blood vessels has passed through the imaging area, or after recirculation and permeation through a tissue portion are performed and the contrast agent contained in the imaging area has been brought to a stationary state.

Thereafter, the operator inputs designation information of a first time range using the first time range designation key 26 from the input unit 107 while referring to the B-mode image information stored in the image memory unit 104 (Step S503). Here, the first time range corresponds to a time range included in the imaging time range. The time taken to administer the contrast agent and thereafter start the penetration of the contrast agent lying in the blood vessels into the corresponding imaging area of a B-mode image is defined as the start time. The end time is set after the contrast agent lying in the blood vessels have passed through the imaging area. The first time range is set as the time range that includes all permeation processes in which the contrast agent in the imaging area changes, as will be described later. Incidentally, the designation information of the first time range is set using information indicative of the order of acquiring frame numbers of B-mode image information or the like provided in time with the time required to acquire the B-mode image information or the acquisition time.

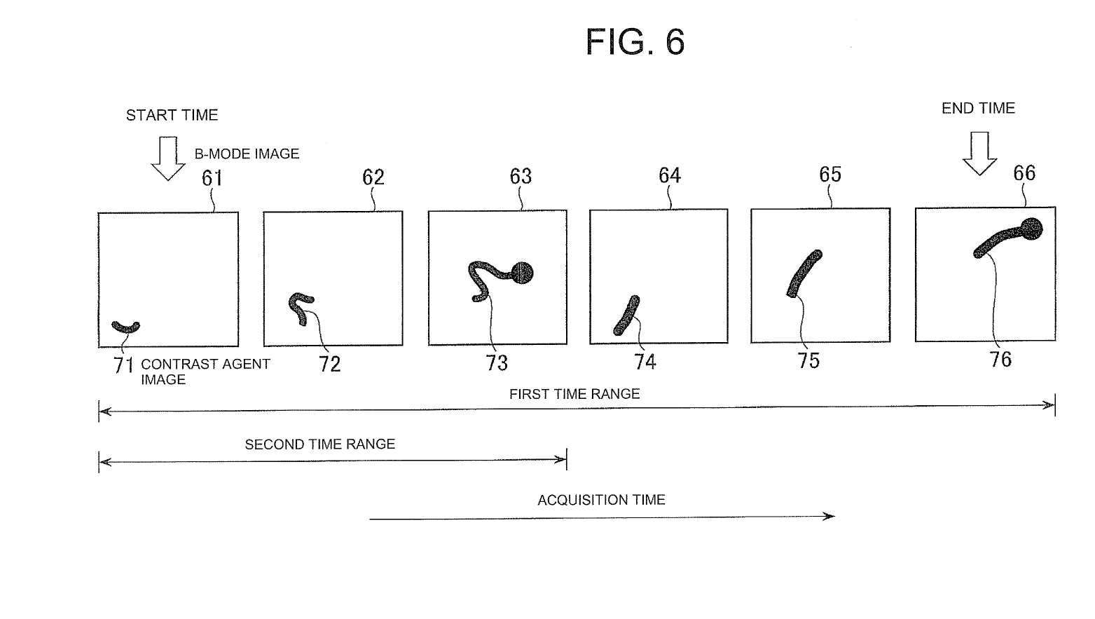

FIG. 6 is an explanatory diagram in which only image information that belong to the set first time range are taken out from the B-mode image information stored in the image memory unit 104 and B-mode images 61 through 66 of theses image information are arranged in accordance with acquired time order. The B-mode 61 is an image at the start time of the first time range. In the B-mode image 61, a contrast agent image 71 lying in the blood vessels, which has begun to penetrate in the imaging area, exists in the lower left of the imaging area. Subsequently, the contrast agent image 71 circulates in the blood vessels and performs such migrations as indicated by contrast agent images 72 through 76 of the B-mode images 62 through 66. The B-mode image 66 is an image at the end time of the first time range. The contrast agent image 71 located at the lower left of the B-mode image 61 upon the start time is one moved to the upper right. Incidentally, FIG. 6 shows the B-mode images existent in the first time range in simplified form. In practice, the number of frames indicative of the number of images exceeds a few hundred of frames, and the B-mode images 61 through 66 also result in ones fragmented in time.

Referring back to FIG. 5, the operator thereafter inputs designation information of a second time range using the second time range designation key 27 from the input unit 107 while referring to the B-mode image information stored in the image memory unit 104 (Step S504). The second time range is a time range that indicates in-progress time during which the contrast agent in the imaging area changes. As shown in FIG. 6, for example, the second time range has the same start time as that of the first time range and has an end time in the course of the first time range.

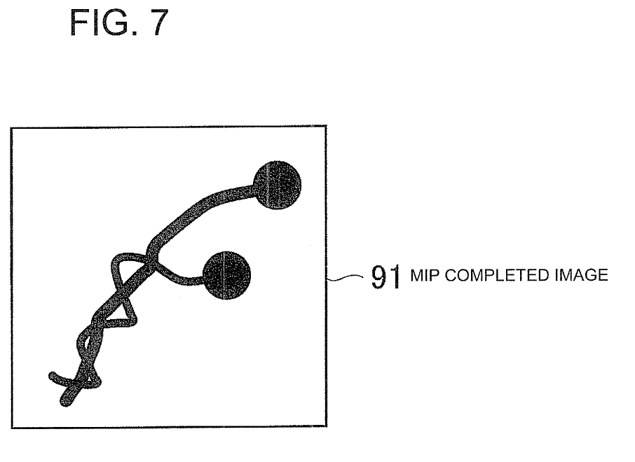

Thereafter, the controller 108 forms MIP completed image information from the B-mode images 61 through 66 in the first time range using the first maximum intensity projection device 81 (Step S505). FIG. 7 is an explanatory diagram showing an MIP completed image 91 of the MIP completed image information formed from the B-mode images 61 through 66. The MIP completed image 91 is an image obtained such that all of the contrast agent images 71 through 76 corresponding to high-brightness areas or regions contained in the B-mode images 61 through 66 are all overlaid. The MIP completed image 91 is an image indicative of all migration processes of the contrast agent moved within the blood vessels in the imaging area.

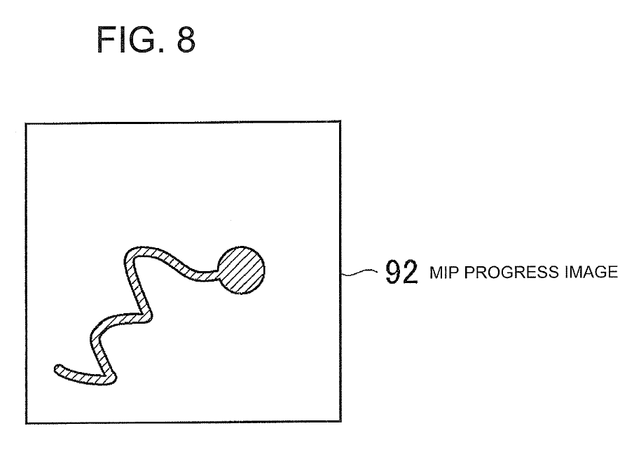

Subsequently to the above, the controller 108 forms MIP elapsed or progress image information corresponding to the maximum intensity projection image of the B-mode images in the second time range using the second maximum intensity projection device 82 (Step S506). FIG. 8 is a diagram showing an MIP elapsed or progress image 92 of the MIP progress image information formed by the second maximum intensity projection device 82 where the range of the B-mode images 61 through 63 is designated as the second time range. The MIP progress image 92 is an image obtained by combining the contrast agent images 71 through 73 corresponding to the high-brightness regions contained in the B-mode images 61 through 63 and is shown as a diagonally-shaded area in the figure.

Then, the operator determines whether when the MIP completed image 91 and the MIP progress image 92 are displayed in overlapped form, the MIP progress image 92 should be displayed in color (Step S507). When the MIP progress image 92 is not displayed in color (NO at Step S507), the operator inputs overlay ratio information from the input unit 107 (Step S509).

When the MIP progress image 92 is displayed in color (YES at Step S507), the operator selects such a contrast table 85 as shown in FIG. 4 in which the pixel values and the hues are associated with one another (Step S508). The second image formation device 83 forms a MIP elapsed or progress image with a code indicative of the hue as a pixel value from the MIP progress image 92 corresponding to the second image (Step S510). Incidentally, the MIP progress image is an image whose diagonally-shaded portion is displaced in color, when the MIP progress image 92 shown in FIG. 8 is used.

Thereafter, the overlay ratio changing device 86 of the overlaid image generation device 84 adjusts the pixel values of the MIP progress image 92 and the MIP completed image corresponding to the first image, based on the overlay ratio information and performs the generation and display of the overlaid image at the same position on the screen of the display unit 106 (Step S511), after which the present processing is terminated. Incidentally, when the MIP progress image 92 is set as the image displayed in color, the overlaid image generation device 84 can also omit the adjustment using the overlay ratio information or replace the image at the position where the MIP completed image 92 is displayed in color with the MIP progress image 92.

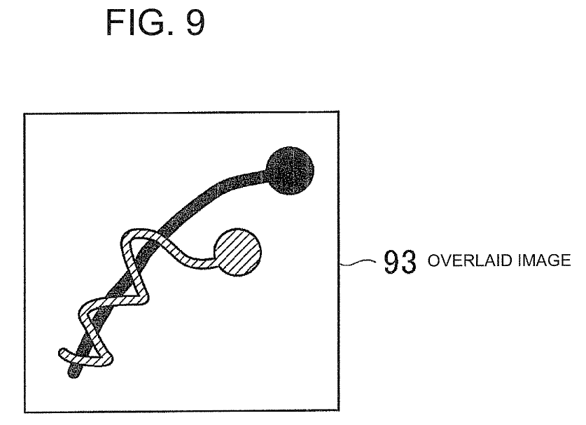

FIG. 9 is a diagram showing an overlaid image 93 formed such that the MIP progress image 92 is overlaid on the MIP completed image 91. The overlaid image 93 is obtained such that the MIP progress image 92 is overlaid on the MIP completed image 91. In FIG. 9, the portion indicative of the MIP progress image 92 is diagonally shaded to make it easy to understand a distinction from the MIP completed image 91. The MIP progress image 92 is displayed with the MIP completed image 91 as the background. Thus, the operator is able to perform a contradistinction between the MIP progress image 92 and the MIP completed image 91 indicative of all permeation processes for the contrast agent.

In the present embodiment as described above, the MIP completed image 91 corresponding to the first image is formed using the B-mode images 61 through 66 acquired in the first time range from the start of the permeation of the contrast agent through the imaging area to its end. The MIP progress image 92 corresponding to the second image is formed using the B-mode images 61 through 63 acquired in the second time range contained in the first time range. The overlaid image 93 with the MIP progress image 92 overlaid thereon is generated with the MIP completed image 91 as the background image, and the overlaid image 93 is displayed. Therefore, the contradistinction between the MIP progress image 92 indicative of the contrast-agent permeation process and the MIP completed image 91 indicative of all permeation processes for the contrast agent is easily performed, by extension, the change in the MIP progress image 92 is made easy to understand.

While the MIP progress image is represented by the diagonally-shaded portion in FIGS. 8 and 9 in the present embodiment, the MIP progress image is displayed as an area different in brightness or hue from the MIP completed image 91 at the actual display unit 106. That is, the overlaid image generation device 84 forms the MIP progress image of the brightness value corresponding to the overlay ratio information set at Step S509, or the second image formation device 83 forms the MIP progress image having the hue code of the contrast table, which has been selected at Step S509.

In the present embodiment, the second maximum intensity projection device 82 and the second image formation device 83 form the single sheet of MIP progress image 92 projected at the maximum value or intensity using the B-mode images 61 through 63 of the second time range as shown in FIGS. 8 and 9. However, the second maximum intensity projection device 82 is also capable of continuously increasing the time range within the second time range in incremental steps, whereby a plurality of MIP progress images that change stepwise within this time range, are overlaid on the corresponding MIP completed image to perform cine display which represents the so-overlaid one as a moving picture or image.

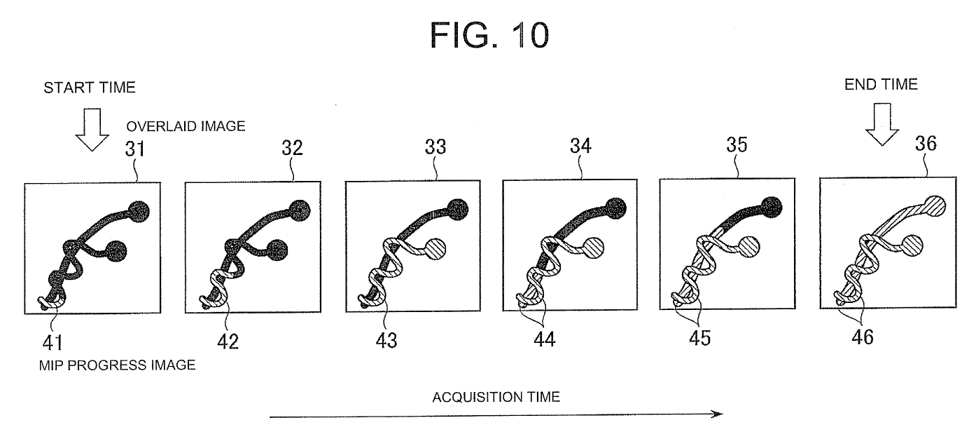

FIG. 10 is an explanatory diagram showing one example of the cine display using the B-mode images 61 through 66 shown in FIG. 6. The present example has showed the case in which a second time range that constitutes a time range for the cine display has been caused to coincide with a first time range. The second maximum intensity projection device 82 and the second image formation device 83 form a plurality of MIP elapsed or progress images 41 through 46 increased in time range continuously from the start times of the first and second time ranges to their end times. FIG. 10 shows a plurality of overlaid images 31 through 36 obtained such that these MIP progress images 41 through 46 are overlaid on the MIP completed image 91. The overlaid images 31 through 36 are cine-displayed on the display unit 106 at time intervals each similar to the acquisition time and make it easy to dynamically understand a permeation process of a contrast agent.

Although the present embodiment has shown the case in which the second time range set within the first time range is one, a plurality of second time ranges can also be set within the first time range. In this case, a single sheet of overlaid image is formed using a plurality of MIP elapsed or progress images obtained every plural second time ranges. In order to distinguish between the MIP progress images on the overlaid image, they can be formed as MIP progress images different in hue using hue's contrast tables different every plural second time ranges.

Many widely different embodiments of the invention may be configured without departing from the spirit and the scope of the present invention. It should be understood that the present invention is not limited to the specific embodiments described in the specification, except as defined in the appended claim.

1. An ultrasonic imaging apparatus comprising:
an image acquisition device configured to administer a contrast agent to a subject and to acquire B-mode image information in an imaging area of the subject through which the contrast agent permeates;
an image storing device configured to store the B-mode image information that changes according to the permeation of the contrast agent during a predetermined imaging time range from after the administration of the contrast agent;
a first maximum intensity projection device configured to form first maximum intensity projection image information by maximum intensity projection using the B-mode image information acquired in a first time range contained in the imaging time range;
a second maximum intensity projection device configured to form second maximum intensity projection image information by maximum intensity projection using the B-mode image information acquired in a second time range contained in the first time range;
a second image formation device configured to form a second image based on the second maximum intensity projection image information;
an overlaid image generation device configured to generate an overlaid image in which the second image is overlaid on a first image based on the first maximum intensity projection image information; and
a display device configured to display the overlaid image.

2. The ultrasonic imaging apparatus according to claim 1, further comprising an input unit configured to input designation formation on the imaging time range, the first time range, and the second time range.

3. The ultrasonic imaging apparatus according to claim 2, wherein said input unit comprises a time range designation key configured to input the designation information on the imaging time range, the first time range, and the second time range.

4. The ultrasonic imaging apparatus according to claim 3, wherein said time range designation key is configured to input one of frame number information and acquisition time information of the B-mode image information constituting the designation information while the B-mode image information for the imaging time range is displayed on said display device.

5. The ultrasonic imaging apparatus according to claim 2, wherein the designation information includes start time information and end time information for the imaging time range.

6. The ultrasonic imaging apparatus according to claim 2, wherein the designation information includes start time information and end time information for the first time range.

7. The ultrasonic imaging apparatus according to claim 2, wherein the designation information includes start time information and end time information for the second time range.

8. The ultrasonic imaging apparatus according to claim 1, wherein the second image is based on the second maximum intensity projection image information.

9. The ultrasonic imaging apparatus according to claim 1, wherein said second image formation device comprises a contrast table in which pixel values and hues of image information are associated with one another.

10. The ultrasonic imaging apparatus according to claim 9, wherein the second image is a colour image in which a pixel value of the second maximum intensity projection image information is associated with a hue, based on the contrast table.

11. The ultrasonic imaging apparatus according to claim 1, wherein said input unit comprises an overlay ratio designation key configured to input overlay ratio information indicative of a ratio in brightness between the first image and the second image at the execution of overlay.

12. The ultrasonic imaging apparatus according to claim 11, wherein said overlaid image formation device is configured to change pixel values of the first and second images to overlay the first and second images, based on the overlay ratio information.

13. The ultrasonic imaging apparatus according to claim 1, wherein the second time range includes a plurality of time ranges set with respect to the first time range.

14. The ultrasonic imaging apparatus according to claim 13, wherein said second image formation device comprises a plurality of contrast tables, each of said plurality of contrast tables including different hues, said second image formation device configured to associated said plurality of contrast tables with one another every second maximum intensity projection image information for the set time ranges in order to form second images different in hue.

15. The ultrasonic imaging apparatus according to claim 1, wherein said second maximum intensity projection device is configured to change the second time range with time stepwise from zero to form second maximum intensity projection image information at each step.

16. The ultrasonic imaging apparatus according to claim 8, wherein said second maximum intensity projection device is configured to change the second time range with time stepwise from zero to form second maximum intensity projection image information at each step.

17. The ultrasonic imaging apparatus according to claim 11, wherein said second maximum intensity projection device is configured to change the second time range with time stepwise from zero to form second maximum intensity projection image information at each step.

18. The ultrasonic imaging apparatus according to claim 15, wherein said second image formation device is configured to form a second image based on the second maximum intensity projection image information for each step, said overlaid image formation device is configured to generate an overlaid image using the second image for each step.

19. The ultrasonic imaging apparatus according to claim 18, wherein said display device is configured to cine-display the overlaid image for each step in sync with the change with the time.

20. An ultrasonic imaging apparatus comprising:
an image acquisition device configured to administer a contrast agent to a subject and to acquire B-mode image information in an imaging area of the subject through which the contrast agent permeates;
an image storing device configured to store the B-mode image information that changes according to the permeation of the contrast agent during a predetermined imaging time range from after the administration of the contrast agent;
a first maximum intensity projection device configured to form first maximum intensity projection image information by maximum intensity projection using the B-mode image information acquired in a first time range contained in the imaging time range;
a second maximum intensity projection device configured to change a second time range contained in the first time range with time stepwise from zero and to form second maximum intensity projection image information by maximum intensity projection using the B-mode image information acquired in the second time range for each step;
a second image formation device configured to form a second image based on the second maximum intensity projection image information at each step;
an overlaid image generation device configured to generate an overlaid image in which the second image is overlaid on a first image based on the first maximum intensity projection image information at each step; and
a display device configured to display the overlaid image at each step.
